SEQUENCE LISTING (6 sequences)

SEQ ID NO: 1 - RNA, 22 nt, Artificial Sequence
  Probe
ucaccggguguaaaucagcuug

SEQ ID NO: 2 - RNA, 24 nt, Artificial Sequence
  Probe
uacccguaaucuucauaauccgag

SEQ ID NO: 3 - RNA, 23 nt, Artificial Sequence
  Probe
uuuguacuccgaugccauucaga

SEQ ID NO: 4 - RNA, 22 nt, Artificial Sequence
  Probe
uucaaguaauccaggauaggcu

SEQ ID NO: 5 - RNA, 22 nt, Artificial Sequence
  Probe
cucaguagccaguguagauccu

SEQ ID NO: 6 - DNA, 21 bp, Artificial Sequence
  Adaptor
tggaattctcgggtgccaagg

(19) United States
(12) Patent Application Publication
(10) Pub. No.: US 2014/0272993 A1
(43) Pub. Date: Sep. 18, 2014

(54) METHOD OF SEQUENCING A FULL MICRORNA PROFILE FROM CEREBROSPINAL FLUID

(71) Applicant: THE TRANSLATIONAL GENOMICS RESEARCH INSTITUTE, Phoenix, AZ (US)

(72) Inventors: Kendall Van Keuren-Jensen, Phoenix, AZ (US); Kasandra L. Burgos, Phoenix, AZ (US)

(73) Assignee: THE TRANSLATIONAL GENOMICS RESEARCH INSTITUTE, Phoenix, AZ (US)

(21) Appl. No.: 14/214,927

(22) Filed: Mar. 15, 2014

Related U.S. Application Data
(60) Provisional application No. 61/794,099, filed on Mar. 15, 2013.

(51) Int. Cl. C12Q 1/68

(52) U.S. Cl. CPC C12Q 1/6806
USPC 435/6.12

(57) ABSTRACT

The present invention provides methods of purifying RNA from a biological sample comprising two separate aqueous extractions of the organic phase obtained by mixing the biological sample with a first solution comprising guanidinium isothiocyanate and beta-mercaptoethanol and a second solution comprising phenol, chloroform, and isoamyl alcohol. Also provided are methods of sequencing RNA purified from a biological sample and methods of diagnosing Alzheimer's disease and Parkinson's disease in a subject by determining whether a plurality of miRNAs has deregulated expression in biological sample from the subject.

### CROSS-REFERENCE TO RELATED APPLICATIONS

The present application claims priority to U.S. Application No. 61/794,099, filed Mar. 15, 2013, the entire contents and disclosure of which are herein incorporated by reference thereto.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 1,356 byte ASCII (text) file named “Seq_List” created on Mar. 15, 2014.

### TECHNICAL FIELD

This application relates to methods of efficiently purifying small RNAs from a biological sample and of sequencing these small RNAs with Next Generation Sequencing (NGS). Also provided are methods of diagnosing Alzheimer's and Parkinson's disease in a subject by measuring the expression of a plurality of microRNAs from a biological sample from the subject.

### BACKGROUND

Scientists looking to perform next-generation sequencing (NGS) must consider the manner and method of sample preparation. The way that DNA or RNA is isolated from tissue, the preparation chosen to construct sequencing libraries, and the type of sequencing that is being performed, all become crucial factors in the experimental design (Baudhuin L. M. (2013) Quality guidelines for next-generation sequencing. Clin Chem 59 858-859).

For RNA sequencing in particular, classes of molecules are, at least in part, defined and sequenced by their size. MicroRNAs (miRNAs; 16-27 nucleotides (nt)), small interfering RNAs (siRNAs; 16-27 nt), and PIWI interacting RNAs (piRNA; ˜30 nt) are all part of a class of small non-coding RNA involved in sequence-specific gene silencing (Castel S. E., Martienssen, R. A. (2013) RNA interference in the nucleus: roles for small RNAs in transcription, epigenetics and beyond. Nat 14, 100-112). While currently known as the smallest functional class, the depth of small RNA's biological significance to regulate gene expression is still being uncovered some 15 years after discovery (Fire A., Xu S., Montgomery M. K., Kostas, et al. (1998) Potent and specific genetic interference by double-stranded RNA in Caenorhabditis Elegans. Nat 391, 806-811.)

Until recently, methods for isolating RNA from tissues of origin had been thought to recover all RNA species. Roughly from large to small, RNA as a family of molecules includes coding RNA (mRNA), long noncoding RNA (lncRNA), transfer RNA (tRNA), small nucleolar RNA (snoRNA), PIWI Interacting RNA (piRNA), and miRNA (Castel S. E., Martienssen, R. A. (2013) RNA interference in the nucleus: roles for small RNAs in transcription, epigenetics and beyond. Nat 14, 100-112.) The purification of all species of RNA is implied in the description of many commercially available kits and methods touting “total” RNA isolation. In fact, it had been used for methods that do not recover small RNA at all, such as column-based kits that washed the small RNA off the column during the cleaning steps. In addition, other kits used ratios of salt and alcohol that are too low to precipitate small RNA out of solution. There are now many commercially available kits for small RNA purification from which to choose. Systematic testing shows that the performance of RNA extraction kits varies quite a bit depending on the type of sample. Reasonably, different kits may deal with a particular sample type better than another. For example, a fibrous tissue such as muscle has to be handled differently than lipid-rich nervous tissue. When available, the best option may be to choose a kit specifically designed to deal with the challenges of a particular type of tissue. There is a need to identify methods to maximize the amount of RNA extracted from biological samples with any given extraction kit especially when limited material is available as is the case with cerebrospinal fluid (CSF).

The discovery and reliable detection of markers for neurodegenerative disease has been complicated by the inaccessibility of the diseased tissue and the inability to biopsy or test tissue from the central nervous system directly. RNAs derived from hard to access tissues, such as neurons within the brain and spinal cord, have the potential to get to the periphery where they can be detected non-invasively. The formation and release of extracellular microvesicles and RNA binding proteins have been found to carry RNA from cells of the central nervous system to the periphery and protect the RNA from degradation. Extracellular miRNAs detectable in peripheral circulation can provide information about cellular changes associated with human health and disease. In order to associate miRNA signals present in cell-free peripheral biofluids with neurodegenerative disease status of patients with neurodegenerative diseases such as Alzheimer's disease (AD) and Parkinson's disease (PD), there is a need to assess the miRNA content in CSF and serum (SER) from subjects with full neuropathological evaluations and to identify those miRNA with deregulated expression levels that correlate with the presence and severity of neurodegenerative disease.

### SUMMARY

The present invention provides a method of purifying RNA from a biological sample, the method comprising: a) mixing the biological sample with a first solution comprising guanidinium isothiocyanate and beta-mercaptoethanol to generate a first mixture; b) combining a second solution comprising phenol, chloroform, and isoamyl alcohol with the first mixture; c) centrifuging the first mixture to produce a first aqueous phase, an interface, and an organic phase; d) removing and saving the first aqueous phase; e) mixing nuclease-free water with the organic phase and the interface to generate a second mixture; f) centrifuging the second mixture to produce a second aqueous phase with the interface and the organic phase; g) removing and saving the second aqueous phase with the first aqueous phase; and h) concentrating and purifying RNA from the first and second aqueous phases with ethanol-based column chromatography or ethanol precipitation and solubilization of the RNA.

In certain embodiments, the acidic pH is between about 1 and about 6, between about 2 and about 6, between about 3 and about 6, between about 4 and about 6, or between about 5 and about 6. In one embodiment, the pH is between about 4 about 5.4. In another embodiment, the method further comprises measuring the volume of the first aqueous phase and mixing nuclease-free water with the organic phase in a volume that is about equal to the volume of the first aqueous phase.

In other embodiments, the second solution consists of 50% phenol, 48% chloroform, and 2% isoamyl alcohol. The second solution may be mixed with the first mixture in a ratio of 1:1 (v/v), 1:2 (v/v), 1:3 (v/v), 2:1 (v/v), or 3:1 (v/v).

In another aspect, the present invention relates to a method of sequencing RNA in a biological sample, the method comprising: a) purifying the RNA from the biological sample with the methods disclosed herein; and b) sequencing the RNA with next-generation sequencing (NGS). The RNA may be any one of siRNA, miRNA, piRNA, gRNA, snoRNA, and tRNA.

Biological samples that may be extracted and sequenced with the methods disclosed herein include, but are not limited to, CSF, whole blood, SER, plasma, urine, saliva, synovial fluid, a bronchioalveolar lavage, a nasal swab, brain tissue, cardiac tissue, bone, skin, a lymph node tissue, and a dental tissue.

In some embodiments, NGS comprises ion semiconductor sequencing, cycle sequencing, pyrosequencing, or sequencing using γ-phosphate-labeled nucleotides.

The present invention also provides a method for diagnosing AD in a subject, the method comprising: a) obtaining a biological sample from the subject; b) determining the expression level of a plurality of miRNAs in the biological sample; and c) detecting AD in the subject if there is a significant deregulation of the expression levels of the plurality of miRNAs in the biological sample compared to control values.

In some embodiments the biological sample is CSF, and the plurality of miRNAs comprises at least two miRNAs selected from the group consisting of miR-124-3p, miR-138-5p, miR-127-3p, miR-132-3p, miR-127-5p, miR-136-3p, miR-381, miR-101-5p, miR-199b-5p, miR-136-5p, miR-184, miR-181a-5p, miR-598, miR-218-5p, miR-9-3p, miR-769-5p, miR-95, miR-760, miR-181a-3p, miR-181b-5p, miR-488-3p, miR-495, miR-708-3p, miR-874, miR-873-5p, miR-129-5p, miR-181d, miR-139-5p, miR-3200-3p, miR-431-3p, miR-9-5p, miR-326, miR-377-5p, miR-433, miR-323a-3p, miR-134, miR-329, miR-10a-5p, miR-33b-5p, miR-410, and miR-708-5p; and the significant deregulation of the expression levels of the plurality of miRNAs is a decrease in expression compared to control values, which is indicative of AD.

In other embodiments, the biological sample is SER, and the plurality of miRNAs comprises at least two miRNAs selected from the group consisting of miR-34b-3p, miR-219-2-3p, miR-22-5p, miR-125b-1-3p, miR-1307-5p, miR-34c-5p, miR-34b-5p, miR-887, miR-135a-5p, miR-184, miR-30c-2-3p, miR-873-3p, miR-125a-3p, miR-671-3p, miR-1285-3p, miR-3176, and miR-127-3p; and the significant deregulation of the expression levels of the plurality of miRNAs is a decrease in expression compared to control values, which is indicative of AD. Alternatively, the biological sample is SER, and the plurality of miRNAs comprises at least two miRNAs selected from the group consisting of miR-182-5p, miR-21-5p, miR-375; and the significant deregulation of the expression levels of the plurality of miRNAs is an increase in expression compared to control values, which is indicative of AD.

The present invention also provides a method for diagnosing Parkinson's disease in a subject, the method comprising: a) obtaining a biological sample from the subject; b) determining the expression level of a plurality of miRNAs in the biological sample; and c) detecting Parkinson's disease in the subject if there is a significant deregulation of the expression levels of the plurality of miRNAs in the biological sample compared to control values.

In some embodiments, the biological sample is CSF, and the plurality of miRNAs comprises at least two miRNAs selected from the group consisting of miR-132-5p, miR-485-5p, miR-127-3p, miR-128, miR-409-3p, miR-433, miR-370, miR-431-3p, miR-873-3p, miR-136-3p, miR-212-3p, miR-10a-5p, miR-1224-5p, and miR-4448; and the significant deregulation of the expression levels of the plurality of miRNAs is a decrease in expression compared to control values, which is indicative of PD. Alternatively, the biological sample is CSF, and the plurality of miRNAs comprises at least two miRNAs selected from the group consisting of miR-19a-3p, miR-19b-3p, and let-7g-3p; and the significant deregulation of the expression levels of the plurality of miRNAs is an increase in expression compared to control values, which is indicative of PD.

In yet other embodiments, the biological sample is SER, and the plurality of miRNAs comprises miR-16-2-3p and miR-1294; and the significant deregulation of the expression levels of the plurality of miRNAs is a decrease in expression compared to control values, which is indicative of PD. Alternatively, the biological sample is SER, and the plurality of miRNAs comprises at least two miRNAs selected from the group consisting of miR-338-3p, miR-30e-3p, and miR-30a-3p; and the significant deregulation of the expression levels of the plurality of miRNAs is an increase in expression compared to control values, which is indicative of PD.

### BRIEF DESCRIPTION OF THE DRAWINGS

FIG. 1 depicts the work flow for first and second extractions. (A) The RNA and denaturing solution are mixed with phenol-chloroform and centrifuged. (B) The aqueous phase is removed and placed in a fresh tube. (C) RNase-free water equal to the volume of the aqueous phase that was removed is added back to the residual interphase and organic layers. (D) Solution is mixed and centrifuged. (E) The aqueous layer is removed and placed into a clean tube as Extraction 2.

FIG. 2 shows that repeated extraction of the organic phase results in higher RNA yield. (A) Fresh-frozen plasma from two subjects (subject 1 and subject 2) was used for RNA isolation using the top four kits: mirVana and mirVana PARIS (Ambion), miRNeasy (Qiagen), and BiooPure (BiooScientific). Total RNA was recovered and quantified from repeated extractions (black=Extraction 1 and gray=Extraction 2). PARIS kit yielded the highest amount of RNA from both subjects. The yield was more than doubled by the second extraction. (B) Fresh-frozen CSF samples from two subjects were used to compare the efficiency of the top four RNA isolation kits. The RNA recovered in Extraction 1 and Extraction 2 is displayed.

FIG. 3 shows miRNA yields calculated from plasma and CSF with repeated extractions using qRT-PCR. (A) miRNA recovered in Extraction 1 was measured by TaqMan qRT-PCR in fresh-frozen plasma samples from two subjects (subject 1 and subject 2). Crossing point values (Cp) were compared across three different synthetic C. elegans miRNA cel-238, cel-54, and cel-39 (spike-ins) and two endogenous human miRNA hsa-222 and hsa-26A. The lowest Cp values indicate the highest amount of RNA present and best performance, highlighted by the black line. (B) Extraction 2 recovery of miRNA is displayed for each kit. (C) The Cp values for two different subject CSF samples for Extraction 1. There was only enough RNA remaining after RiboGreen for cel-238. (D) Cp values for cel-238 recovered from two CSF samples in Extraction 2.

FIG. 4 presents the top 50 most abundant miRNAs identified in human CSF with the RNA extraction methods described herein followed by NGS.

FIG. 5 shows potential sources of variation for the sample cohort. Three-way ANOVA analysis of variation demonstrates that (A) expiration age, (B) postmortem interval (PMI) and (C) gender do not contribute significant variation to the miRNA expression data.

FIG. 6 shows differentially expressed miRNAs detected in the CSF.

FIG. 7 shows differentially expressed miRNAs detected in the SER.

### DETAILED DESCRIPTION

As used herein, the verb “comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”.

As used herein, the term “subject” or “patient” refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In other embodiments, the subject is a human.

As used herein the term “diagnosing” or “diagnosis” refers to the process of identifying a medical condition or disease by its signs, symptoms, and in particular from the results of various diagnostic procedures, including e.g. detecting the expression of the nucleic acids according to at least some embodiments of the invention in a biological sample obtained from an individual. Furthermore, as used herein the term “diagnosing” or “diagnosis” encompasses screening for a disease, detecting a presence or a severity of a disease, distinguishing a disease from other diseases including those diseases that may feature one or more similar or identical symptoms, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations. The diagnostic procedure can be performed in vivo or in vitro.

“Detection” as used herein refers to detecting the presence of a component (e.g., a nucleic acid sequence) in a sample. Detection also means detecting the absence of a component. Detection also means measuring the level of a component, either quantitatively or qualitatively. With respect to the method of the invention, detection also means identifying or diagnosing Alzheimer's disease or Parkinson's disease in a subject. “Early detection” as used herein refers to identifying or diagnosing Alzheimer's disease or Parkinson's disease in a subject at an early stage of the disease (e.g., before the disease causes symptoms).

“Differential expression” as used herein refers to qualitative or quantitative differences in the temporal and/or cellular expression patterns of a transcript within and among cells and tissue. Thus, a differentially expressed transcripts can qualitatively have its expression altered, including an activation or inactivation, in, e.g., normal versus disease tissue. Genes, for instance, may be turned on or turned off in a particular state, relative to another state thus permitting comparison of two or more states. A qualitatively regulated gene or transcript may exhibit an expression pattern within a state or cell type that may be detectable by standard techniques. Some transcripts will be expressed in one state or cell type, but not in both. Alternatively, the difference in expression may be quantitative, e.g., in that expression is modulated, up-regulated, resulting in an increased amount of transcript, or down-regulated, resulting in a decreased amount of transcript. The degree to which expression differs need only be large enough to quantify via standard characterization techniques such as expression arrays, quantitative reverse transcriptase PCR, northern analysis, and RNase protection.

In some embodiments, the term “level” refers to the expression level of a miRNA according to at least some embodiments of the present invention. Typically the level of the miRNA in a biological sample obtained from the subject is different (e.g., increased) from the level of the same miRNA in a similar sample obtained from a healthy individual (examples of biological samples are described herein). Alternatively, the level of the miRNA in a biological sample obtained from the subject is different (e.g., increased) from the level of the same miRNA in a similar sample obtained from the same subject at an earlier time point. Alternatively, the level of the miRNA in a biological sample obtained from the subject is different (e.g., increased) from the level of the same miRNA in a non-diseased tissue obtained from said subject. Typically, the expression levels of the miRNA of the invention are independently compared to their respective control level.

The term “expression level” is used broadly to include a genomic expression profile, e.g., an expression profile of miRNAs. Profiles may be generated by any convenient means for determining a level of a nucleic acid sequence e.g. quantitative hybridization of miRNA, labeled miRNA, amplified miRNA, cDNA, etc., quantitative PCR, ELISA for quantitation, and the like, and allow the analysis of differential gene expression between two samples. A subject or tumor sample, e.g., cells or collections thereof, e.g., tissues, is assayed. Samples are collected by any convenient method, as known in the art. According to some embodiments, the term “expression level” means measuring the abundance of the miRNA in the measured samples.

The plurality of miRNAs described herein, optionally includes any sub-combination of markers (i.e., miRNAs), and/or a combination featuring at least one other marker, for example a known marker. As described herein, the plurality of markers is preferably then correlated with the presence or stage of a disease. For example, such correlating may optionally comprise determining the concentration of each of the plurality of markers, and individually comparing each marker concentration to a threshold level. Optionally, if the marker concentration is above the threshold level, the marker concentration correlates with Alzheimer's disease or Parkinson's disease. Optionally, a plurality of marker concentrations correlates with Alzheimer's disease or Parkinson's disease. Alternatively, such correlating may optionally comprise determining the concentration of each of the plurality of markers, calculating a single index value based on the concentration of each of the plurality of markers, and comparing the index value to a threshold level. Also alternatively, such correlating may optionally comprise determining a temporal change in at least one of the markers, and wherein the temporal change is used in the correlating step.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers is outside of a normal range, this subset may be indicative of a particular diagnosis/prognosis. The skilled artisan will also understand that diagnostic markers, differential diagnostic markers, prognostic markers, time of onset markers, disease or condition differentiating markers, etc., may be combined in a single assay or device. Markers may also be commonly used for multiple purposes by, for example, applying a different threshold or a different weighting factor to the marker for the different purpose(s).

In the methods of the invention, a “significant elevation” in expression levels of the plurality of miRNAs refers, in different embodiments, to a statistically significant elevation, or in other embodiments to a significant elevation as recognized by a skilled artisan. For example, without limitation, the present invention demonstrates that an increase of about at least two fold, or alternatively of about at least three fold, of the threshold value is associated with Alzheimer's disease or Parkinson's disease.

In additional embodiments, a significant elevation refers to an increase in the expression of a plurality of miRNAs.

The term “about” as used herein refers to +/−10%.

Diagnostic methods differ in their sensitivity and specificity. The “sensitivity” of a diagnostic assay is the percentage of diseased individuals who test positive (percent of “true positives”). Diseased individuals not detected by the assay are “false negatives”. Subjects who are not diseased and who test negative in the assay are termed “true negatives”. The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

In one embodiment, the method distinguishes a disease or condition (particularly cancer) with a sensitivity of at least 70% at a specificity of at least 70% when compared to normal subjects (e.g., a healthy individual not afflicted with cancer). In another embodiment, the method distinguishes a disease or condition with a sensitivity of at least 80% at a specificity of at least 90% when compared to normal subjects. In another embodiment, the method distinguishes a disease or condition with a sensitivity of at least 90% at a specificity of at least 90% when compared to normal subjects. In another embodiment, the method distinguishes a disease or condition with a sensitivity of at least 70% at a specificity of at least 85% when compared to subjects exhibiting symptoms that mimic disease or condition symptoms.

Diagnosis of a disease according to at least some embodiments of the present invention can be affected by determining a level of a polynucleotide according to at least some embodiments of the present invention in a biological sample obtained from the subject, wherein the level determined can be correlated with predisposition to, or presence or absence of the disease (i.e., Alzheimer's disease or Parkinson's disease).

The term “sample” or “biological sample” as used herein means a sample of biological tissue or fluid or an excretion sample that comprises nucleic acids. Such samples include, but are not limited to, tissue or fluid isolated from subjects. Biological samples may also include sections of tissues such as biopsy and autopsy samples, frozen sections, blood, plasma, SER, sputum, stool and mucus. Biological sample also refers to metastatic tissue obtained from, but not limited to, organs such as liver, lung, and peritoneum. Biological samples also include explants and primary and/or transformed cell cultures derived from animal or patient tissues. Biological samples may also be blood, a blood fraction, gastrointestinal secretions, or tissue sample. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used.

In some embodiments the sample obtained from the subject is a body fluid or excretion sample including but not limited to seminal plasma, blood, SER, urine, prostatic fluid, seminal fluid, semen, the external secretions of the skin, respiratory, intestinal, and genitourinary tracts, tears, CSF, sputum, saliva, milk, peritoneal fluid, pleural fluid, peritoneal fluid, cyst fluid, lavage of body cavities, broncho alveolar lavage, lavage of the reproductive system and/or lavage of any other organ of the body or system in the body, and stool.

Numerous well known tissue or fluid collection methods can be utilized to collect the biological sample from the subject in order to determine the expression level of the biomarkers of the invention in said sample of said subject.

Examples include, but are not limited to, blood sampling, urine sampling, stool sampling, sputum sampling, aspiration of pleural or peritoneal fluids, fine needle biopsy, needle biopsy, core needle biopsy and surgical biopsy, and lavage. Regardless of the procedure employed, once a biopsy/sample is obtained the level of the biomarkers can be determined and a diagnosis can thus be made. Tissue samples are optionally homogenized by standard techniques e.g. sonication, mechanical disruption or chemical lysis. Tissue section preparation for surgical pathology can be frozen and prepared using standard techniques. In situ hybridization assays on tissue sections are performed in fixed cells and/or tissues.

In a one embodiment, blood is used as the biological sample. If that is the case, the cells comprised therein can be isolated from the blood sample by centrifugation, for example.

As used herein, the terms “nucleic acid” and “polynucleotide” are used interchangeably, and include polymeric forms of nucleotides of any length, either deoxyribonucleotides or ribonucleotides, or analogs thereof. The following are non-limiting examples of polynucleotides: a gene or gene fragment, exons, introns, messenger RNA (mRNA), microRNA transfer RNA (tRNA), ribosomal RNA (rRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The term also includes both double- and single-stranded molecules.

miRNAs are a large class of single strand RNA molecules of 18-25 nucleotides, involved in post transcriptional gene silencing. Eighty percent of conserved miRNA show tissue-specific expression and play an important role in cell fate determination, proliferation, and cell death (Lee and Dutta. Annu. Rev. Pathol. Mech. Dis. 2009; 4: 199-227; Ross, Carlson and Brock, Am J Clin Path 2007: 128; 830-836). miRNAs arise from intergenic or intragenic (both exonic and intronic) genomic regions that are transcribed as long primary transcripts (pri-microRNA) and undergo a number of processing steps to produce the final short mature molecule (Massimo et al., Current Op. in Cell Biol. 2009: 21; 1-10).

The mature miRNAs suppress gene expression based on their complementarity to a part of one or more mRNAs usually in the 3′ UTR site. The annealing of miRNA to the target transcript either blocks protein translation or destabilizes the transcript and triggers the degradation or both. Most of the miRNA action on target mRNA translation is based on the partial complementarity, therefore conceivably one miRNA may target more than one mRNA and many miRNAs may act on one mRNA (Ying at el., Mol. Biotechnol. 2008: 38; 257-268). In humans, approximately one-third of miRNAs are organized into clusters. A given cluster is likely to be a single transcriptional unit, suggesting a coordinated regulation of miRNAs in the cluster (Lee and Dutta. ibid).

There are a number of considerations when choosing protocols both upstream and downstream of NGS experiments. On the front end, purification methods, additives, and residuum can often inhibit the sensitive chemistries by which sequencing-by-synthesis is performed. On the back end, data handling, analysis software packages, and pipelines can also impact sequencing outcomes. The present invention provides methods of preparing biological samples (e.g., acellular biofluid samples) for small RNA sequencing.

In one embodiment, the present invention provides that in regards to purification methods small RNA yield can be improved considerably by following the total RNA isolation protocol included with Ambion's mirVana PARIS kit but modifying the organic extraction step. Specifically, after transferring the upper aqueous phase to a fresh tube, water is added to the residual material (interphase and lower organic layer) and again phase-separated. In contrast, all the protocols provided with the commercially available kits at the time of the invention required only one organic extraction. This simple yet, as it turns out, quite useful modification allows access to previously inaccessible material. Potential benefits from these changes are a more comprehensive sample profiling of small RNA, as well as wider access to small volume samples, such as acellular biofluids, which now can be prepared for small RNA sequencing on the Illumina platform.

In one embodiment, the present invention provides methods of sequencing the full profile of miRNA from a biological sample (e.g., plasma or CSF). The inventors have now examined differentially expressed miRNAs identified in Alzheimer's and Parkinson's patients and during different the development of different disease pathologies. miRNAs that are significantly differentially expressed between Alzheimer's disease or Parkinson's disease patients and controls, during pathogenesis, as well as miRNAs that are differentially expressed between Alzheimer's and Parkinson's patients.

In certain aspects, the present invention provides a method of obtaining enough RNA from biofluid samples to do miRNA sequencing. With the prior art methods it was difficult to obtain enough RNA from the biofluid samples to do miRNA sequence. As described herein, the inventors provide methods and markers for Alzheimer's disease or Parkinson's disease, as the expression of the miRNAs change with disease severity. The method and markers are useful as diagnostics to identify patients at high risk for disease and requiring intervention.

The present invention also provides for the sequencing of miRNA from CSF and plasma from the same individuals. The miRNAs are useful as markers for Alzheimer's disease or Parkinson's disease, as the expression of the miRNAs change with disease severity. Commercial value resides in the ability to use the markers as diagnostics to identify patients at high risk for disease and requiring intervention. Biomarkers for neurodegenerative diseases are in high demand to help identify the patients that need to be treated and when. Applicant provides for the first time sequencing data on these miRNAs from biofluids that are useful in therapeutics and diagnostics. In certain embodiments, one or more of the isolated miRNAs are part of a diagnostic device or kit.

In some embodiments, the purified RNA from the biological sample is analyzed by Sequencing by Synthesis (SBS) techniques. SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in some of the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides. In methods using nucleotide monomers lacking terminators, the number of different nucleotides added in each cycle can be dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.). In preferred methods a terminator moiety can be reversibly terminating.

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.). However, it is also possible to use the same label for the two or more different nucleotides present in a sequencing reagent or to use detection optics that do not necessarily distinguish the different labels. Thus, in a doublet sequencing reagent having a mixture of A/C both the A and C can be labeled with the same fluorophore. Furthermore, when doublet delivery methods are used all of the different nucleotide monomers can have the same label or different labels can be used, for example, to distinguish one mixture of different nucleotide monomers from a second mixture of nucleotide monomers. For example, using the [First delivery nucleotide monomers]+[Second delivery nucleotide monomers] nomenclature set forth above and taking an example of A/C+(1/T), the A and C monomers can have the same first label and the G and T monomers can have the same second label, wherein the first label is different from the second label. Alternatively, the first label can be the same as the second label and incorporation events of the first delivery can be distinguished from incorporation events of the second delivery based on the temporal separation of cycles in an SBS protocol. Accordingly, a low resolution sequence representation obtained from such mixtures will be degenerate for two pairs of nucleotides (T/G, which is complementary to A and C, respectively; and C/A which is complementary to G/T, respectively).

Some embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. No. 6,210,891; U.S. Pat. No. 6,258,568 and U.S. Pat. No. 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons.

In another example type of SBS, cycle sequencing is accomplished by stepwise addition of reversible terminator nucleotides containing, for example, a cleavable or photobleachable dye label as described, for example, in U.S. Pat. No. 7,427,67, U.S. Pat. No. 7,414,1163 and U.S. Pat. No. 7,057,026, the disclosures of which are incorporated herein by reference. This approach is being commercialized by Solexa (now Illumina Inc.), and is also described in WO 91/06678 and WO 07/123,744 (filed in the United States Patent and Trademark Office as U.S. Ser. No. 12/295,337), each of which is incorporated herein by reference in their entireties. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

In other embodiments, Ion Semiconductor Sequencing is utilized to analyze the purified RNA from the sample. Ion Semiconductor Sequencing is a method of DNA sequencing based on the detection of hydrogen ions that are released during DNA amplification. This is a method of “sequencing by synthesis,” during which a complementary strand is built based on the sequence of a template strand.

For example, a microwell containing a template DNA strand to be sequenced can be flooded with a single species of deoxyribonucleotide (dNTP). If the introduced dNTP is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple dNTP molecules will be incorporated in a single cycle. This leads to a corresponding number of released hydrogens and a proportionally higher electronic signal.

This technology differs from other sequencing technologies in that no modified nucleotides or optics are used. Ion semiconductor sequencing may also be referred to as ion torrent sequencing, proton-mediated sequencing, silicon sequencing, or semiconductor sequencing. Ion semiconductor sequencing was developed by Ion Torrent Systems Inc. and may be performed using a bench top machine. Rusk, N. (2011). “Torrents of Sequence,” Nat Meth 8(1): 44-44. Although it is not necessary to understand the mechanism of an invention, it is believed that hydrogen ion release occurs during nucleic acid amplification because of the formation of a covalent bond and the release of pyrophosphate and a charged hydrogen ion. Ion semiconductor sequencing exploits these facts by determining if a hydrogen ion is released upon providing a single species of dNTP to the reaction.

For example, microwells on a semiconductor chip that each contain one single-stranded template DNA molecule to be sequenced and one DNA polymerase can be sequentially flooded with unmodified A, C, G or T dNTP. Pennisi, E. (2010). “Semiconductors inspire new sequencing technologies” Science 327(5970): 1190; and Perkel, J., “Making contact with sequencing's fourth generation” Biotechniques (2011). The hydrogen ion that is released in the reaction changes the pH of the solution, which is detected by a hypersensitive ion sensor. The unattached dNTP molecules are washed out before the next cycle when a different dNTP species is introduced.

Beneath the layer of microwells is an ion sensitive layer, below which is a hypersensitive ISFET ion sensor. All layers are contained within a CMOS semiconductor chip, similar to that used in the electronics industry. Each released hydrogen ion triggers the ISFET ion sensor. The series of electrical pulses transmitted from the chip to a computer is translated into a DNA sequence, with no intermediate signal conversion required. Each chip contains an array of microwells with corresponding ISFET detectors. Because nucleotide incorporation events are measured directly by electronics, the use of labeled nucleotides and optical measurements are avoided.

An example of a Ion Semiconductor Sequencing technique suitable for use in the methods of the provided disclosure is Ion Torrent sequencing (U.S. Patent Application Numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and are attached at a resolution such that the fragments are individually resolvable. Addition of one or more nucleotides releases a proton (H+), which signal detected and recorded in a sequencing instrument. The signal strength is proportional to the number of nucleotides incorporated. User guides describe in detail the Ion Torrent protocol(s) that are suitable for use in methods of the invention, such as Life Technologies' literature entitled “Ion Sequencing Kit for User Guide v. 2.0” for use with their sequencing platform the Personal Genome Machine™ (PCG).

In some embodiments, as a part of the sample preparation process, “barcodes” may be associated with each sample. In this process, short oligos are added to primers, where each different sample uses a different oligo in addition to a primer.

The term “library”, as used herein refers to a library of genome-derived sequences. The library may also have sequences allowing amplification of the “library” by the polymerase chain reaction or other in vitro amplification methods well known to those skilled in the art. The library may also have sequences that are compatible with next-generation high throughput sequencers such as an ion semiconductor sequencing platform.

In certain embodiments, the primers and barcodes are ligated to each sample as part of the library generation process. Thus during the amplification process associated with generating the ion amplicon library, the primer and the short oligo are also amplified. As the association of the barcode is done as part of the library preparation process, it is possible to use more than one library, and thus more than one sample. Synthetic DNA barcodes may be included as part of the primer, where a different synthetic DNA barcode may be used for each library. In some embodiments, different libraries may be mixed as they are introduced to a flow cell, and the identity of each sample may be determined as part of the sequencing process. Sample separation methods can be used in conjunction with sample identifiers. For example a chip could have 4 separate channels and use 4 different barcodes to allow the simultaneous running of 16 different samples.

The present invention is further illustrated by the following examples that should not be construed as limiting. The contents of all references, patents, and published patent applications cited throughout this application, as well as the Figures, are incorporated herein by reference in their entirety for all purposes.

### Examples

### Example 1

### Evaluation of RNA Extraction Kits and Protocol Improvements

We tested different commercially available RNA extraction kits and found that some of them were more efficient at isolating small RNA from biofluids than others. Common protocol changes that produced a higher yield of RNA were also tested in all kits. The best conditions to obtain high small RNA yield from cell-free biofluids are outlined in this Example, and these conditions are important to researchers looking to perform small RNA NGS. The current protocol was specifically developed and tested for small RNA isolation from human plasma, SER, and CSF for the purposes of Illumina-based NGS (Illumina, San Francisco, Calif., USA). It has since been further applied to human saliva and urine samples. This method potentially expands the sample types and amounts used for human small RNA profiling.

From among the top four kits for isolation of total and small RNA, MaxRecovery BiooPure RNA Isolation Reagent (Bioo Scientific, Austin, Tex., USA) was not selected because the invisible final pellet caused some loss of RNA in some samples, and the miRNeasy kit (Qiagen, Valencia, Calif., USA) was not selected either because it has an 18 nt lower size limit cutoff for RNA recovery, precluding 67 of 2578 or ˜2.6% of all mature miRNAs (mirBase: the microRNA Database [Internet]. Release 20. Manchester (England): University of Manchester. 2006; updated 2013 Jun. 24). The standard mirVana kit (Life Technologies), which does not offer researchers the option for protein isolation from the original lysate, performed well but was not chosen because the first buffer is added at 10 times the sample volume. Therefore, more than 50 individual centrifugation steps would be required for each 1 mL of sample, making this method logistically unreasonable for biofluid RNA isolation. The mirVana PARIS (Protein and RNA Isolation) Kit (Life Technologies) performed the best for RNA yield, ease, and application when systematically compared with the other commercially available kits and methods (Burgos K. L. Javaherian A. Bomprezzi R. Ghaffari L. et al. (2013) Identification of extracellular miRNA in human cerebrospinal fluid by next-generation sequencing. RNA 5, 712-722.)

The mirVana PARIS miRNA purification kit includes use of a proprietary lysis buffer with β-mercaptoethanol which serves to denature biofluid proteins, an acidic phenol:chloroform extraction to isolate RNA from the protein, lipid, and DNA content, followed by an alcohol/column-based cleaning step before RNA elution. In this Example, we describe an off-label method for optimized miRNA extraction from acellular biofluids. The main changes are in addition to the standard protocol provided by the manufacturer, and include re-extracting RNA from, instead of disposing of, the organic residual phenol:chloroform by adding a volume of water, remixing, and separating another aqueous volume. These changes are summarized in the Methods section of this Example from step 3.3.9 to step 3.3.11. Although the level of improvement in small RNA yield using the modifications proposed in this Example may vary depending upon the particular kit this method is applied to, it has been shown to have cross platform applicability (Burgos K. L. Javaherian A. Bomprezzi R. Ghaffari L. et al. (2013) Identification of extracellular miRNA in human cerebrospinal fluid by next-generation sequencing. RNA 5, 712-722.). Kits using a phenol:chloroform RNA isolation may benefit by adding the extra steps that we used for the mirVana PARIS kit. The RNA yield from all kits that were tested benefitted from a second aqueous extraction from the phenol:chloroform residual material.

A notable finding was the best kits for recovery of large RNA molecules (quantified fluorometrically using Quant-iT Ribogreen RNA, Life Technologies) were not the best for recovery of small RNA (quantified by TaqMan qRT-PCR, Life Technologies). In fact, of the top 4 kits in each category of either the best small RNA recovery or the best large RNA recovery, only two kits were shared across them; therefore, some kits recovered one size RNA better than another. Hence, this Example will focus on the description of methods that will enable researchers to maximize small RNA recovery. Since current methods of NGS on small RNA are performed separately from large RNA, the fact that the best kits for extraction of small or large RNA molecules are different does not pose an issue at the time.

The method described here was tested and shown to improve small RNA recovery from plasma, SER, and CSF. However, this method is not limited to these sample types and can reasonably be applied to other types of acellular biofluids. In addition, the Illumina Small RNA Sample Preparation Kit and Illumina HiSeq 2000 were used for NGS downstream of the purification (Life Technologies).

The following protocol provides one embodiment of the present invention. This protocol is capable of further modifications and this application is intended to cover any variations, uses, or adaptations of the invention following, in general, the principles of the invention and including such departures from the present disclosure as come within known or customary practice within the art to which the invention pertains.

### 1. Materials

1. Ambion mirVana PARIS Kit (see Note 1): miRNA Wash Solution 1, Wash Solution 2/3 (see Note 2), Collection Tubes and Filter Cartridges (see Note 3), Cell Disruption Buffer (see Note 4), 2× Denaturing Solution, Acid-Phenol:Chloroform (see Note 5), Elution Solution (see Note 6).
2. 200-proof ethanol (ethyl alcohol), ACS grade or better (see Note 7).
3. β-mercaptoethanol.
4. 7 M ammonium acetate.
5. 2 mL cryovial (for sample).
6. Bench-top centrifuge capable of at least 800×g.
7. Biosafety cabinet.
8. Fume-hood with negative air-flow (see Note 8).
9. Large centrifuge capable of maintaining room temperature and centrifuging at least 10,000×g using a rotor able to hold 15 mL conical tubes (see Note 9).
10. Laboratory heating block set to 95-100° C.
11. Rocking or rotating platform (see Note 10).
12. RNase-free low-bind 1.5 mL polypropylene microfuge tubes (see Note 11).
13. RNase decontamination wipes or spray (see Note 12).

### 2. Methods

2.1 Sample Handling
Once the biofluid is collected from the host, flash-freeze 1 mL in a 2 mL cryovial either in liquid nitrogen or in a dry-ice/200-proof-ethanol slurry to preserve the RNA profile (see Note 13). Use of a biosafety cabinet is required when handling biological samples to protect researchers from human pathogen exposure.

2.2 Prepare Kit Solutions
1. Allow mirVana PARIS kit to come to room temperature (see Note 14).
2. Add 21 mL 100% ethanol to miRNA Wash Solution 1 (see Note 7).
3. Add 40 mL of 100% ethanol to Wash Solution 2/3 (see Notes 7 and 15).
4. Add 375 μL β-mercaptoethanol to 2× Denaturing Solution (see Note 16).
5. Aliquot 1 mL of nuclease-free molecular biology grade water (see Note 6) into 1.5 mL microfuge tubes, and place them on heating block set to 95° C. This pre-heated water will be used to elute RNA from the column in the final step (see Note 17).

2.3 Modified mirVana PARIS miRNA Isolation Protocol
1. Add an equal volume of 2× Denaturing Solution to frozen biofluid sample (see Note 18).
2. Place sample on a rocking or rotating platform at room temperature until fully thawed and mixed (see Note 10).
3. Incubate at room temperature for 10 minutes.
4. Add an equal volume of Acid-Phenol:Chloroform (see Note 19).
5. Vortex for 30 seconds to mix.
6. Centrifuge at 10,000×g for 5 minutes at room temperature (see Notes 20).
7. Carefully remove the tubes from the centrifuge, and check that there is an upper (aqueous) layer and a lower (organic) layer.
8. Transfer approximately 90% of the upper aqueous phase of this first extraction to a clean tube and estimate the volume. Take care to leave behind a volume of aqueous liquid so that the meniscus does not touch the interphase (see Note 21). Set aside.
9. To the left over organic residuum, add a volume of water equivalent to the aqueous volume that was just transferred to the new tube.
10. Vortex for 30 seconds to mix.
11. Centrifuge at 10,000×g for 5 minutes at room temperature.
12. Transfer approximately 90% of the upper aqueous phase of this second extraction to the same tube that contains the first aqueous volume removed from the phenol chloroform (see Note 21). The remainder of the phenol:chloroform can now be discarded (see Note 5).
13. Add 1.5× volumes of 100% ethanol to the total aqueous volume removed from first and second organic extractions (see Note 7).
14. Invert 10 times to mix, and let solution stand at room temperature for 10 minutes.
15. Apply solution through column, 700 μL at a time, by centrifugation at not more than 800×g (see Note 22), discarding flow-through at each pass, and reassemble filter column and reservoir tube (see Note 3).
16. Apply 700 μL of prepared Wash Solution 1 to the column (see Note 23), and centrifuge at 800×g for 30 seconds to pass solution through filter column (see Note 22). Discard flow-through, and reassemble filter column and reservoir tube (see Note 3).
17. Apply 500 μL of prepared Wash Solution 2/3 to the column (see Note 24), and centrifuge at 800×g for 30 seconds to pass solution through filter column. Discard flow-through, and reassemble filter column and reservoir tube.
18. Repeat step 17.
19. Without applying any other solutions, centrifuge filter column and empty reservoir tube for 30 seconds to dry residual ethanol.
20. Transfer filter column to fresh tube (see Note 25).
21. Apply 100 μl of 95° C. (see Note 26) nuclease-free water (see Note 6) to the filter column, and incubate at room temperature for 1 min.
22. Centrifuge filter column at 10,000×g for 1 minute to elute RNA from the column (see Note 27).
23. Repeat step 22-23.
24. The filter component of the column assembly can be discarded as RNA has been eluted from the filter and is in the flow-through in the collection tube.
25. Centrifuge RNA sample at maximum speed for 1 min to collect residual column fibers.
26. Avoiding the residual fibers from the filter column, transfer the RNA sample to a new microfuge tube. Proceed to ethanol precipitation for small RNA NGS sample preparation (see Note 27).
27. Add 0.5 volumes 7 M ammonium acetate to a final concentration of 2-2.5 M. Mix well (see Note 28).
28. Add 4 volumes of 100% ethanol. Mix well, and place at −20° C. from 4 hours to overnight.
29. Centrifuge at 16,000×g for 30 min at 4° C. to precipitate RNA (see Note 29).
30. Wash pellet twice with 80% ethanol (see Note 30).
31. Resuspend RNA pellet in volume of water as downstream protocol dictates.

### 3. Notes

1. The mirVana PARIS kit is enough for 40 reactions when using the manufacturer provided-protocol and suggested tissues (see Ambion mirVana PARIS user guide). With the modified protocol described here, one 40 reaction kit will purify ˜20 mL of biofluid.
2. Wash Solution 2/3 is used for the second and third rinse of the silica-based column containing the immobilized RNA.
3. The filter column and collection tube will be reused at all steps in this modified protocol, with the exception of the last one where the RNA isolation and purification is complete.
4. Cell Disruption Buffer is included in the reagent list, however will not be used for the current method that was designed for cell-free biofluid samples.
5. The Acid-Phenol:Chloroform is caustic; therefore, care must be taken during the handling and disposal. Personal protective equipment and the use of a fume hood is required.
6. Elution Solution is provided for final elution of the RNA for routine purposes. In the current protocol, nuclease-free molecular biology grade water is used for elution of the RNA.
7. As the ratio of ethanol to aqueous buffer is important to whether or not RNA is dissolved in—or precipitating out—of solution, it is crucial to use 200-proof, ACS grade or better, ethanol in making the alcohol:buffer solutions. Each time dehydrated ethanol is exposed to the environment, water from atmospheric humidity will dissolve in it, subsequently decreasing the ethanol content of the downstream solution. Using a small bottle of 200-proof ethanol, or aliquoting a larger bottle into smaller volumes, will increase the likelihood that the ethanol remains as the stock.
8. For safety reasons, with the exception of the last step, the entire protocol should be performed in a fume-hood with negative airflow designed for volatile chemicals.
9. The pH of all buffers and solutions is an important aspect of their molecular function. Since temperature has a significant effect on pH, it should be controlled. All steps described here are done at room temperature unless otherwise stated. However, extended centrifugation may increase the temperature of the sample being centrifuged. Therefore, the centrifuges used in the non-column-based centrifugation steps must be set to the standard ambient temperature of 25° C. For brief centrifugation steps, such as the ones for passing liquid through microfuge columns, a temperature-controlled centrifuge is not required.
10. It is not important at which speed a standard laboratory rocking or rotating platform is used as long as it allows a thorough mixing of the frozen biofluid in the denaturing buffer.
11. We found that the collection tubes supplied with the mirVana PARIS kit did not always tightly cap. In addition, the use of low-binding tubes decreases evaporation and residual RNA material left behind in the storage tube. Therefore, once the RNA has been eluted from the column, it should be transferred to a tightly capped nuclease-free low-binding microfuge tube.
12. Clean bench and all equipment that will be used for RNA purification with RNase decontamination spray or wipes according to the manufacturer's recommendation for those products. Overall precaution should be taken to minimize possible exposure of RNA to RNAases.
13. While miRNA has been shown to be relatively stable, treating samples the same way each time will ensure that collection bias is minimized, and will preserve the total RNA profile. In frozen samples, RNases are inactive due to the low temperature that does not allow water to be in the liquid form necessary for these proteins to degrade RNA. Samples are thawed in the presence of 2× Denaturing Solution to ensure that RNases are denatured; therefore, they are irreversibly inactivated.
14. The mirVana PARIS Kit is shipped at room temperature, and components are either stored at room temperature or at 4° C. according to the manufacturer's specifications. For either the routine use or the current modified protocol, the mirVana Paris kit components should be allowed to come to room temperature before use.
15. A white precipitate of excess EDTA might form in the Wash Solution 2/3 but it is of no consequence and should be left behind in the bottle when using this solution.
16. The 2× Denaturing Solution forms a precipitate at the recommended storage temperature of 4° C. Once warmed to room temperature, visually inspect the solution. If a solid white precipitate is present, place the bottle tightly closed at 37° C. and, occasionally, mix until solution is fully reconstituted.
17. Microfuge-tube cap locks or aluminum foil can be used to ensure the tubes stay closed under increased temperature and pressure from the evaporating solution.
18. Estimate the volume of the biological sample. If the sample tube is more than halfway full, which would prevent that an equal volume of 2× Denaturing Solution be added, add only 1/10th volume of 2× Denaturing Solution in the tube, and mix vigorously until frozen sample is slightly loosened from the tube. Transfer frozen sample and residual solution to a larger tube that has the remaining 2× Denaturing Solution.
19. A small volume of aqueous buffer overlays the organic Acid-Phenol:Chloroform. When using this reagent, be sure that two distinct layers are present. Agitation of this solution should be avoided so that the layers do not mix. If the solution looks cloudy or small bubbles are present, it should be allowed to settle until the two layers are visibly separate. When using this solution, be sure to withdraw Acid-Phenol:Chloroform from beneath the aqueous buffer layer. When the volume of solution gets low, be sure to watch that you are withdrawing the Acid-Phenol:Chloroform and not the overlying buffer.
20. The phenol-chloroform phase separation steps involve centrifuging a relatively large volume. Therefore, it is advisable that the rotor for the temperature-regulated centrifuge (see Note 6) is confirmed to be compatible with centrifuge tubes that can hold this volume prior to beginning the purification procedure. The tube should be capable of holding 5 times the volume.
21. Depending on the biofluid, a white interphase may or may not be obvious, particularly for the second extraction. Upon careful inspection, the phases should be visible and should not be disrupted when pipetting the upper aqueous volume.
22. The columns from the mirVana PARIS kit were designed for the manufacturer's protocol. With the modified method, larger volumes than originally intended pass though the column. As RNA will bind to the fibers of the column, it is best to carefully maintain the integrity of the column. Therefore, the maximum centrifugation speed recommended for passing the aqueous extraction/ethanol solution is 800×g.
23. Prepared Wash Solution 1 contains 21 mL 100% ethanol.
24. Prepared Wash Solution 2/3 contains 40 mL 100% ethanol.
25. To prevent dried residual material from being introduced into the fresh reservoir tubes, clean the outside of the filter column using a wipe with 70% ethanol solution but avoid wetting the filter.
26. Pre-heat an aliquot of nuclease-free molecular biology grade water on a heat block set to 95° C., and use it to elute RNA from the filter column. To account for evaporation at this temperature, double the volume that will be used should be pre-heated
27. If the RNA will be used for any other sequencing aside from small RNA, DNAse treatment of the sample may be necessary.
28. Ethanol precipitation of RNA should always proceed with the salt being added to the RNA sample and thoroughly mixed prior to adding alcohol.
29. Centrifuge the tube with the hinge of the cap out so that the RNA collects under the hinge inside the tube. As the RNA will likely be translucent at this stage, it will be easier to locate and avoid disrupting.
30. Be sure to allow 80% ethanol to run down the hinge side of the interior of the microfuge tube.

### Example 2

### Experimental Materials and Methods

The following materials and methods were used for Examples 3 and 4.

### Clinical Samples

All clinical samples included in the current study were obtained from subjects who had given informed consent, and studies were performed under the guidelines of Institutional Review Board (IRB)-approved protocols at St. Joseph's Hospital and the Translational Genomics Research Institute (TGen).

Patient plasma, SER, and CSF samples were obtained. Blood draws were performed from the antecubital veins directly into Vacutainer potassium EDTA tubes (BD Vacutainer) as a routine part of the neurological workup. Within 2 h of the blood draw, samples were processed for plasma or SER isolation. CSF was obtained by lumbar puncture, and samples were spun down to pellet cells, and the supernatant removed and flash-frozen in liquid nitrogen for subsequent RNA isolation.

As a preface to this study, to ensure systematic comparison between different RNA purification methods, the plasma samples were thawed on ice, pooled, separated into 200-μL aliquots, flash frozen in liquid nitrogen, and stored at −80° C. until the initial denaturant for the respective kit was added. Each RNA extraction method was tested in triplicate for each kit and/or variation using these 200-mL plasma samples as starting material.

### RNA Extractions

Ten commercially available kits were compared in the current study for the purification of biofluids: BiooPure (BiooScientific), mirVana (Ambion), mirVana PARIS (Ambion), TRI Reagent RT (MRC), TRI Reagent RT-Blood (MRC), TRI Reagent RT-Liquid Samples (MRC), RNAzol (MRC), miRNeasy (Qiagen), and PureLink microRNA (Invitrogen). One of the kits, mirPremier (Sigma), was not found suitable for purifying biofluids as the initial lysate was unable to pass through the column.

For all extractions, we first followed the manufacturer-provided protocol with minor modifications. RNA purifications were performed on virtually identical samples (see clinical samples above) in triplicate for each kit and were rehydrated as called for by the commercially available protocol.

All purifications were performed at room temperature unless a protocol specified a different temperature. For all nine kits, we followed the protocol for total RNA isolation that included recovery of small RNA. In the case of the MRC kits, the protocol allowed for a range of temperatures and centrifugation speeds; the upper and lower limits of those parameters were tested. RNA purifications were performed and quantified side-by-side in triplicate for each kit.

Where applicable, reserved for procedures involving phenol-chloroform phase separation, we rehydrated the interphase and organic layer and subsequently re-extracted to maximize recovery of nucleic acids. This procedure was utilized for the following RNA purification methods that relied upon phase separation: BiooPure, mirVana, mirVana PARIS, TRI Reagent RT, TRI Reagent RT-Blood, TRI Reagent RT-Liquid Samples, and miRNeasy. The phenol was extracted a second time with an equal volume of nuclease-free water to obtain residual aqueous material left at the interface. The two extractions were kept separate throughout and assayed independently for total RNA and miRNA content but were combined for downstream sequencing experiments. After column washes, the RNA was rehydrated on the column, and centrifugation allowed the RNA eluate to be collected. The protocol for the MRC kits allowed for incubation temperatures ranging from 4° C. to 25° C. and centrifugation speeds between 4000 g and 12,000 g; the upper and lower limits of those parameters were also tested. All RNA was precipitated and recovered by either centrifugation (pellet) or elution (column) in molecular biology grade, nuclease-free water (Life Technologies) in the volume and temperature recommended by the kit.

### Determination of RNA Yield

Quantification of total RNA yield was determined by Quant-iT RiboGreen RNA reagent (Invitrogen) utilizing the low-range assay in a 200-μL total volume in the 96-well format (Costar). This protocol allows for quantification of 1-50 pg/μL, the linearity of which is maintained in the presence of common post-purification contaminants such as salts, ethanol, chloroform, detergents, proteins, and agarose (Jones U, Yue S T, Cheung C Y, Singer V L. 1998. RNA quantitation by fluorescence-based solution assay: RiboGreen reagent characterization. Anal Biochem 265: 368-374.). Individual samples were assayed in triplicate, and the means were calculated. The three replicates from the same treatment were averaged. We used the low-range assay (1-50 pg/μL) in a 200-μL total volume of working reagent in a 96-well format and read on a plate reader (BioteK Synergy HT).

In order to simplify the quantification of samples processed with different kits and having varying final volumes, we removed half of the eluent from each sample and adjusted the volume to a final volume of 60 μL for every sample. For example, if kit A recommends to elute in 50 μL and kit B recommends elution in 100 μL, 25 μL and 50 μL, respectively, were removed, and each volume was adjusted to a final volume of 60 μL. The concentration in that 60 μL represents half of the recovered RNA and made downstream assays (i.e., loading 1 μL of each sample into the RiboGreen assay) much easier to process and interpret.

### Real-Time RT-PCR

Input RNA was reverse transcribed using a small-scale reaction with the TaqMan miRNA Reverse Transcription Kit using miRNA specific primers, and real-time RT-PCR (qPCR) was performed using TaqMan miRNA-specific stem-loop primers as described previously (Mitchell P S, Parkin R K, Kroh E M, Fritz B R, Wyman S K, Pogosova-Agadjanyan E L, Peterson A, Noteboom J, O'Briant K C, Allen A, et al. 2008. Circulating microRNAs as stable blood-based markers for cancer detection. Proc Natl Acad Sci 105: 10513-10518).

In order for the recovery of RNA across all samples isolated with different kits to be directly comparable, irrespective of the volume in which the RNA was rehydrated, the RNA input into the reverse transcription (RT) was 50% of the total elution volume scaled up to a set volume of 60 μL across all samples. 1.67 μL was added to the reverse transcription mix. The cycle number at which the fluorescence passes a fixed threshold (Cp) is reported. Probe sequences were (supra Mitchell et al. 2008): cel-miR-39: UCACCGGGUGUAAAUCAGCUUG (SEQ ID NO: 1), cel-miR-54: UACCCGUAAUCUUCAUAAUCCGAG (SEQ ID NO: 2), cel-miR-238: UUUGUACUCCGAUGCCAUUCAGA (SEQ ID NO: 3), hsa-miR-26A: UUCAAGUAAUCCAGGAUAGGCU (SEQ ID NO: 4), hsa-miR-222: CUCAGUAGCCAGUGUAGAUCCU (SEQ ID NO: 5).

Synthetically generated C. elegans miRNAs, which lack sequence homology to the current human miRNA database (miRBase V. 16), were utilized in the current study to correlate absolute cycle threshold data generated by qRT-PCR to the number of molecules of that species present, as previously described (supra Mitchell et al. 2008). Briefly, the synthetic oligonucleotides, generated with 5′ phosphate and 3′ hydroxyl groups to match the molecular structure of RISC complex-processed mature miRNAs (Mitchell et al. 2008), have sequence homology to C. elegans miRNAs cel-miR-39, celmiR-54, and cel-miR-238 (miRBase 16; ordered as custom RNA oligonucleotides from IDT). A mix of these miRNAs at 25 fmol each was prepared and flash-frozen in 10-μL aliquots. A volume of 1.5 μL of the mix was added to each sample after RNase inactivation. For determining the maximal C. elegans recovery, we diluted the 1.5-μL spike-in mix equivalent to the final amount tested in the samples. Because half of the isolated RNA content of the samples is diluted in 60 μL, we put half of the spike-in mix in 60 μL (0.75 μL in 60 μL of RNase-free water). In order to make this even more similar to the samples, half was removed and brought up to 60 μL. 1.67 μL was then used in the reverse transcription reaction (5-μL reaction). 28.9 μL of water was added to the cDNA, and 2.25 μL was used in the Taq reaction (as in Mitchell et al. 2008). We used Cp values of up to 35 accurately to score RNA yield as previously reported (Chen L, Yan H X, Yang W, Hu L, Yu L X, Liu Q, Li L, Huang D D, Ding J, Shen F, et al. 2009. The role of microRNA expression pattern in human intrahepatic cholangiocarcinoma. J Hepatol 50: 358-369; Chen Y, Gelfond J A, McManus L M, Shireman P K. 2009. Reproducibility of quantitative RT-PCR array in miRNA expression profiling and comparison with microarray analysis. BMC Genomics 10: 407). CSF samples, because they have so little RNA, were processed for RT and qPCR slightly differently. Half of the eluted volume was put in 60 μL, as in the plasma samples above. The 60-μL sample was then dried down to 6 μL, 1.67 μL went into the RT reaction, and we added 28.9 μL water. We took 2.25 μL of the RT reaction forward into Taq. When we calculate the return of spike-ins for this experiment using just spike-in mix and water, the Cp values are cel-miR-39 (Cp 15.33), cel-miR-54 (Cp 16.65), and cel-miR-238 (Cp 17.79).

### Small RNA Sequencing

Total RNA was purified from a pool of CSF created from six subject samples using the mirVana PARIS kit and the modified protocol as described. The pooled sample was then separated into aliquots of 500, 750, 1000, 1250, and 1500 μL. After elution of RNA in 100 μL of nuclease-free water, the total RNA was precipitated as described by mixing eluate with ammonium acetate to a final concentration of 2 M, adding four volumes of ethanol, chilling overnight at −20° C., then centrifuging at 16,000 g for 30 min, followed by two 80% ethanol washes. RNA was resuspended in 6 μL of water, the entire volume of which was introduced into half of the TruSeq Small RNA Sample reagents, followed by 15 cycles of PCR to amplify the library.

We clustered a single read v3 flow cell and performed small RNA deep sequencing on the HiSeq 2000 using the RNA isolated from the 0.5- to 1.5-mL aliquots of CSF.

### Sequencing Data Analysis

Raw fastq sequences were generated and de-multiplexed using the Illumina CASAVA v1.8 pipeline. The FastQC and FASTX toolkit were used for Quality Check [ensured that fastq reads are in entirely normal (green tick: ≧Q28) range in the QC report] and to preprocess the reads prior to mapping, respectively. The fastx clipper tool was employed to remove the Illumina 3 prime adaptor (TGGAATTCTCGGGTGCCAAGG) (SEQ ID NO: 6) sequences. Post-clipped reads were then run through mirDeep2 analysis Pipeline (Friedlander M R, Mackowiak S D, Li N, Chen W, Rajewsky N. 2012. miRDeep2 accurately identifies known and hundreds of novel microRNA genes in seven animal clades. Nucleic Acids Res 40: 37-52). Sequences were aligned using mapper.pl to Human genome (hg18) and miRBase_v16 and further processed using miRDeep2.pl scripts. The .csv files for miRNA expression from the mirDeep2 outputs were used for the analysis. Reads per million were calculated as follows: Number of sequenced reads/total reads×1,000,000.

### Example 3

### Maximization of RNA Recovery by Repeated Extraction of the Organic Phase

Organic phase separation for nucleic acid purification requires that the upper aqueous phase containing the RNA be carefully removed from the interphase and the lower organic phase. In an effort to isolate the aqueous layer with the least amount of contamination from the interphase material, some residual RNA-containing aqueous solution is ultimately left behind. To maximize RNA recovery, we rehydrated the interphase and the organic phase left behind and re-extracted the phenol-chloroform solution with water (FIG. 1). We hoped this simple procedure would increase both total RNA and the small RNA yield. While this method is not sophisticated, none of the kits suggest adding liquid back to the remaining interphase and organic layers after the first aqueous phase has been removed and performing a second phenol-chloroform extraction. Several of the kits do suggest a second phenol-chloroform extraction of the first aqueous layer that is removed in order to further clean up the RNA and remove contaminants.

After addition of phenol-chloroform and centrifugation, the aqueous layer of the extraction was carefully removed, measured, and set aside (Extraction 1). Instead of discarding the residual interphase and organic layer from the extraction, we added another volume of RNAse-free water (equal to the volume removed in Extraction 1) to the organic layer and repeated the extraction. We mixed the sample once again in the manner specified by each kit, separated the phases again by centrifugation, and carefully removed the aqueous phase again (Extraction 2) (FIG. 1). We continued to process these two extractions in parallel according to the downstream instructions called for by the respective kit.

While we expected some increase in the recovered RNA, we were surprised to find that the total and small RNA yield was substantially improved by the second extraction with water. To illustrate the increase in RNA recovery using two separate phenol-chloroform extractions in our top kit choices, we acquired 800 μL of fresh-frozen plasma aliquots from two different subjects. We separated the plasma into 200-μL aliquots to be tested in each of the four kits and added a known quantity of spike-in C. elegans miRNAs. We also acquired 8 mL of CSF from two different subjects, separated them into 2-mL aliquots, added C. elegans miRNAs, and tested 2 mL in each of the four kits (Ambion mirVana, Ambion PARIS, BiooPure, and Qiagen miRNeasy).

We quantified the RNA yield in Extraction 1 and Extraction 2 separately by RiboGreen assay (FIGS. 2A and 2B). Quantification of RNA in Extraction 2 from plasma indicates that there is still a large amount of RNA that can be recovered by repeating the extraction. In some cases, such as with the PARIS kit, we were able to more than double our total RNA yield by repeating the extraction. For example, plasma total RNA for subject 1 using the PARIS kit was 48.7 ng by combining 23.35 ng from Extraction 1 with 25.35 ng from Extraction 2. CSF total RNA for subject 1 was 15.8 ng by adding 9.2 ng from Extraction 1 to 6.6 ng from Extraction 2, using the PARIS kit.

We really wanted to know if the isolation of small RNA was increased by this method. We compared the yield of small RNA recovered after isolation from plasma using qRT-PCR for the spiked-in C. elegans miRNAs as well as two endogenous human miRNAs in Extraction 1 (FIG. 3A) and Extraction 2 (FIG. 3B). Recovery of small RNA was markedly increased, and in some cases doubled, by the repeated extraction. We tested extractions on the same sample for a third and fourth time, but the recovery of RNA was very low (data not shown). We also tested the recovery of small RNA from CSF using the four best kits. After quantitation of the CSF with RiboGreen in triplicate, there was so little RNA remaining from the CSF samples that we were able to examine the recovery of only one cel miRNA (cel-238) in Extraction 1 (FIG. 3C) and Extraction 2 (FIG. 3D). Again, in the CSF samples, the recovered miRNAs were greatly increased by performing the second extraction.

### Example 4

### miRNA from CSF Sequenced with NGS

In order to determine whether we can use the small amounts of RNA that can be recovered from the volumes of CSF typically given to us by clinical collaborators, we isolated RNA from a range of starting volumes using a pool of CSF. We chose to use CSF because the total RNA and miRNA fraction has not yet been profiled by NGS. While the TruSeq small RNA kit recommends 1 μg of total RNA to start, 1 mL of CSF only yields ˜15-30 ng of total RNA (FIG. 2B).

We thawed ten 1 mL-samples in the presence of 2× denaturing solution from mirVana PARIS, thoroughly mixed the samples together in a pool, isolated the RNA, and aliquoted the CSF in 0.5, 0.75, 1.0, 1.25, and 1.5 mL volumes in duplicate. To maximize yield, we repeated the extraction of the organic layer as before and combined the RNA from the first and second extractions. Since the total and small RNA are almost immeasurable at these starting volumes of CSF, we isolated RNA from each volume and used the entire amount of isolated RNA for sequencing. We followed sample preparation according to the Illumina TruSeq small RNA kit with one alteration. In order to avoid extensive adaptor dimers forming in the library preparation, we reduced the reagents from the Illumina TruSeq small RNA kit by half. This increased our library preparation success rate and decreased the number of adaptor only contaminating sequences.

The number of reads (raw counts) that mapped to known mature miRNAs in miRBase was more than 1 million for each sample tested and ranged from 1,003,030 to 4,849,671 mapped reads. We calculated Spearman rank correlations by comparing the 0.5- to 1.25-mL starting volumes with the 1.5-mL volume. The correlations were >0.95 for miRNAs with more than five counts. We repeated this experiment using RNA isolated with the BiooPure RNA isolation kit, which also performed very well, and attained nearly identical sequencing results for 0.5- to 1.5-mL starting volumes. These data indicate that we can obtain reproducible results from as little as 0.5 mL of human CSF.

The top 50 most abundant miRNA from the pooled CSF samples are presented in FIG. 4. One of the advantages of sequencing the miRNA is the potential to assay all the miRNA present, including novel miRNA. Using miRDeep2 prediction software, we identified potential new miRNAs from the CSF samples.

We discovered that by repeating the phenol-chloroform extraction with RNase-free water, we could increase our detection of miRNA by almost double. It seems reasonable that we might increase our small RNA yield even more by doing a third or fourth extraction. When we tried this, however, we found that the additional extractions resulted in only a modest increase in yield and did not warrant the additional steps and required processing time (data not shown). We found that the combination of the first and second extractions were sufficient for acquiring enough small RNA for downstream sequencing assays.

It is possible to use these sequencing protocols with small but clinically relevant biofluid sample sizes. Using the RNA isolation protocol described here, we were successfully able to use CSF in downstream sequencing assays. It is possible to sequence miRNA from as little as 0.5 mL of CSF using the methods outlined in the current study. To our knowledge, this is the first time the small RNA fraction of CSF has been sequenced. We surveyed our sequencing results from five subjects' CSF alongside the miRNA counts from normal human brain tissue sequenced by (Hua D, Mo F, Ding D, Li L, Han X, Zhao N, Foltz G, Lin B, Lan Q, Huang Q. 2012. A catalogue of glioblastoma and brain microRNAs identified by deep sequencing. Int J Integr Biol 16: 690-699) and (Skalsky R L, Cullen B R. 2011. Reduced expression of brain enriched microRNAs in glioblastomas permits targeted regulation of a cell death gene. PLoS One 6: e24248). There are many miRNAs that reflect expression levels similar to those observed in brain tissue, but there are also some miRNAs that are more abundant in either the CSF or the brain.

For the first time, we present an approach to sequence extracellular miRNA from human CSF. The methods described here can be used to identify extracellular small RNA in small, clinically obtainable volumes of biofluids and plasma from patient samples and even transgenic mouse models of disease. These methods can be applied to identify novel biomarkers or mechanisms of pathology, or to monitor drug efficacy for a variety of diseases including cancer, neurological diseases, and traumatic brain and spinal cord injury. The results of the sequencing experiments demonstrate that sequencing small RNAs from small starting volumes can provide us with robust, reproducible data.

### Example 5

### Experimental Materials and Methods

The following materials and methods were used for Examples 6-8.

### Samples and Patient Data

Ethics Statement—All subjects were enrolled in the Banner Sun Health Research Institute (BSHRI) Brain and Body Donation Program as a whole-body donor and had previously signed informed consent approved by the BSHRI Institutional Review Board (IRB). The TGen Office of Research Compliance approved the use of the banked postmortem samples for this study. We obtained the following three groups of samples that were used for this study: AD (n=67 CSF and n=64 SER), PD (n=65 CSF and n=60 SER), and control (n=70 CSF and n=72 SER) from the Sun Health Research Institute, Sun City, Ariz. Neuropathological verification of the diagnosis was completed and reported for all samples. FIG. 5 displays no significant source of variation in samples due to age, gender, or postmortem interval (PMI). Note the following abbreviations: AD: Alzheimer's disease; PD: Parkinson's disease; CSF: cerebrospinal fluid; SER; serum.

### RNA Isolation and Sequencing

Total RNA was isolated from 1 ml of CSF and 1 ml of SER from each subject as described in supra Burgos et al., 2013. Briefly, the miRVana PARIS kit (Invitrogen) was used with a modified protocol to extract total RNA and maximize miRNA yield. The Illumina TruSeq Small RNA sequencing kit was used for library preparation as previously described supra Burgos et al., 2013. The samples were given individual barcodes up to 48, pooled and loaded on seven lanes of the Illumina HiSeq2000 with one lane of the flowcell used as a control for calculating phasing throughout the run. Each sample was often sequenced on two different flowcells to maximize reads mapped to mature miRNA sequences in miRBase.

### Post-Sequencing Analysis Pipeline

Sequencing data generated by Illumina HiSeq2000 was pre-processed as previously described in (Metpally R, Nasser S, Courtright A, Carlson E, Villa S, et al. (2013) Comparison of analysis tools for miRNA high throughput sequencing using nerve crush as a model. Front Genet. 4: 20) and aligned to the reference with miRDeep2 software as described (supra Friedländer et al., 2011). The sequencing data was processed and de-multiplexed using Illumina's CASAVA (v1.8) pipeline. Quality control checks on raw fastq reads generated by CASAVA were performed by FastQC software. The FASTX toolkit was used for fastq pre-alignment processing, including adapter clipping and read collapsing, for better mapping results. Illumina three prime adapter sequences were removed by the fastx_clipper tool. Clipped reads were used as an input argument for miRDeep2 alignment software. The processing of sequencing data using miRDeep2 consists of three modules. The

Mapper module preforms read preprocessing and alignment to the reference genome. Once aligned, the miRDeep2 module excises genomic regions covered by the sequencing data in order to identify probable secondary RNA structure. Plausible miRNA precursors are evaluated and scored based on their likelihood of being true events. The Quantifier module produces a scored list of known and novel miRNAs with quantification and expression profiling. We used default parameters suggested by the creators of the tool and allowed one single nucleotide variation (SNV). The csv files from miRDeep2 were used for further analysis.

### Statistical Analysis

### Normalization and Quality Control

The miRNA read counts identified by miRDeep2 were normalized using DESeq2 normalization method to account for compositional bias in sequenced libraries and library size. Assuming typical DESeq2 data frame, the method consists of computing a size factor for each sample as the median ratio of the read count over the corresponding row geometric average (Dillies M A, Rau A, Aubert J, Hennequet-Antier C, Jeanmougin M, et al. (2012) A comprehensive evaluation of normalization methods for Illumina high-throughput RNA sequencing data analysis. Brief Bioinform doi:10.1093/bib/bbs046). Raw counts were then divided by the size factor associated with their sample. Under DESeq2 normalization hypothesis, most genes are not differentially expressed (DE), leading to a ratio of 1. Therefore, the size factor for the sample is an estimate of the correction factor that needs to be applied to all read counts of the corresponding column in order to make samples comparable.

Quality control of miRNA expression data consisted of filtering both samples and miRNAs. Samples with total sum of mapped read counts lower than 100,000 for CSF and 60,000 for SER were removed. Thresholds were determined based on the distribution of the total counts for all samples. Additionally, miRNAs with average less than 5 counts were not considered for further analysis.

### Differential Expression

Differential expression of miRNA read counts was performed using DESeq2 (v2.1.0.19) package (Anders S, Huber W. (2010) Differential expression analysis for sequence count data. Genome Biol. 11:R106). Three groups were considered for paired analysis from CSF data: i) Control and Alzheimer's subjects, ii) Control and Parkinson's subjects, and iii) Alzheimer's and Parkinson's subjects. Similarly, three groups were considered for paired analysis from SER data: i) Control and Alzheimer's subjects, ii) Control and Parkinson's subjects, and iii) Alzheimer's and Parkinson's subjects. DESeq2 method is based on negative binomial distribution (NB), with custom fit for variance-mean dependence (supra Anders et al., 2010). Upon normalization, dispersion is estimated by local regression for gamma-family generalized linear models, providing basis for inference. Sum of all replicates for gene i corresponding to conditions A and B, C_{iA} and C_{iB}, are evaluated as NB-distributed with moments as estimated and fitted. The p value of a pair of observed count sums (C_{iA}, C_{iB}) is then the sum of all probabilities less or equal to p(C_{iA}, C_{iB}), conditioned on C_{iA}+C_{iB} (supra Anders et al., 2010). We report differentially expressed miRNA with fold change 0.7<FC(log 2) or FC(log 2)<−0.7 significant at adjusted p-value <0.05.

### Ordinal Logistic Regression

To take advantage of the ordinal nature of regional and time-depended characteristics present in AD and PD pathology, we implemented ordinal logistic regression (OLR) in order to detect miRNAs with monotonic expression patterns. Ordinal logistic model assumes the presence of a covert continuous predictor variable and ordinal outcome that arises from discretization of the underlying continuum into j-ordered groups such that j=[1 . . . J] [75]. Analysis of ordered categorical data was executed via cumulative link models (CLMs). Ordinal response variable Yᵢ then follows multinomial distribution with probability pᵢⱼ that the ith observation falls in response category j. Ordinal logit considers the probability of a single event and all events that are ordered before it, hence incorporating ordered nature of the dependent variable in the fit [75]. With cumulative probabilities set to yᵢⱼ=P(Yᵢ≦j)=pᵢ₁+ . . . +pᵢⱼ, cumulative logits which incorporate the logit link are defined as:

logit(yᵢⱼ)=log((P(Yi≦j)/(1−P(Yᵢ≦j))

j=[1 . . . J−1] (3)

Let Xᵢ be a vector of explanatory variables, β the corresponding set of regression parameters, and αⱼ provides each cumulative logit its unique intercept value. Then, cumulative logit model is a regression model for cumulative logits defined as:

logit(yᵢⱼ)=αⱼ−βXᵢ(4) (4)

Four well described signatures of AD and PD pathology were binned into ordinal categories and considered as OLR outcome variables: i) Braak neurofibrillary stages, ii) neurofibrillary tangle scores, iii) plaque-density scores and iv) synuclein/Lewy body stages. Neuropathological examination disclosed total Braak stages (1-6), neurofibrillary tangle neurofibrillary tangle (0-15), plaque-density scores (1-15) and Lewy body stages (no Lewy bodies; Limbic type; Neocortical type). For convenience, we binned the neurofibrillary tangele and plaque-density scores for each subject into three ordinal categories, in increasing increments. The events of interest correspond to low neurofibrillary tangles score (0-4), moderate neurofibrillary tangles score (5-9) and high neurofibrillary tangles score (10-15). Similarly, for plaque-density data three groups correspond to low plaque-density score (1-5), moderate plaque density score (6-10) and high plaque-density score (11-15). Lastly, synuclein/Lewy body stage was divided into ordinal outcome variables as defined by the Unified Staging System for Lewy Body Disorders corresponding to lowest progression (no Lewy bodies), moderate progression (Limbic type) and advanced progression (Neocortical type) (Beach T G, Adler C H, Lue L, Sue L I, Bachalakuri J, et al. (2009) Unified staging system for Lewy body disorders: correlation with nigrostriatal degeneration, cognitive impairment and motor dysfunction. Acta Neuropathol. 117:613-634).

The OLR method was used to model relationship between the ordinal outcome variables and explanatory predictor variable, namely normalized miRNA counts, using the R package ordinal. Logit build-in link function was used to determine factors associated with Braak, neurofibrillary tangle and plaque density stages. The cumulative link model assumes that thresholds are constant for all values of the explanatory variables. For reported miRNAs, graphical method for assessing the parallel slopes assumption was used to check ordinal logit requirements. A modified Newton algorithm was used to optimize the likelihood function. The condition number of the Hessian did not indicate a problem with any of the models corresponding to reported miRNAs. Parameter confidence intervals were based on the profile likelihood function, and the estimates in the output are given in units of ordered log odds.

Additionally to the usual hypothesis-testing approach, we decided to estimate the effect of a certain variable on the response outcome and its precision. The objective of the model selection analysis is to evaluate whether the effect of the possible predictor is sufficiently important, and as such, is it possible to make predictions based on a regression model that includes it as a parameter. Akaike Information Criterion is a particularly useful information theory approach for model selection when a number of variables are believed to have an effect on a process or a pattern. For the same dataset with the same response variable, the “best” model is the one that minimizes the Kullback-Leibler value, or the information loss when approximating a real process (Kullback S, Leibler R A. (1951) On information and sufficiency. Annals of Mathematical Statistics. 22:79-86). In order to minimize the expected Kullback-Leibler information, it is necessary to maximize E_{y}Eₓ[log (g(x|θ(y))) for a collection of admissible models, where g is the approximated model in terms of a probability distribution, y is the random sample from the density function ƒ(y) for the unknown real process ƒ, and θ is the maximum likelihood estimate based on the model g and data y (supra Kullback et al., 1951). Approximately unbiased maximum likelihood estimate of E_{y}Eₓ[log (g(x|θ(y))) for a large sample corresponds to AIC=−2 logL(θ (y))+2 k, where k is the number of estimated parameters included in the model and logL(θ (y)) is the log-likelihood of the model given the data, which reflects the overall fit of the model (Hurvich C M, Tsai C. (1989) Regression and time series model selection in small samples. Biometrika. 76: 297-307). Essentially, AIC provides an indication of which model would best approximate reality, in terms of minimizing the loss of information, as well as gives a measure of strength of evidence for each model.

For the acquired data, we tested a series of plausible models. The global model, defined as the most complex model considered, was constructed as a set of variables suspected of having an effect on the outcome variable (OLR, uncorrected p-value <0.05, parameter estimate 95% confidence interval did not include zero). Fit of the global model was assessed first. In case of a fit, simpler models, originating from the global model, were compared based on the weight of evidence that model i is the best approximation of the true mathematical model given the data and the set of considered candidates (Burnham K P, Anderson DR. 2002. Model Selection and Multimodel Inference: a practical information-theoretic approach. Springer-Verlag, New York, N.Y.). The value of the AIC has no important meaning unless compared to AIC of a series of alternate models. Note that a small Kullback-Leibler information discrepancy in a model corresponds to a small AIC value for the same model. The AIC differences, Δᵢ, quantify the information loss when one of the fitted models is used instead of the best approximating model. In general, 0≦Δᵢ≦2 suggests substantial evidence for the model, 3≦Δᵢ≦7 indicates the model has considerably less support, whereas Δᵢ>10 signifies that the model is very unlikely due to essentially no support (supra Burnham et al., 2002). We considered predictor variables significant at unadjusted p-value <0.05 and Δᵢ≦10.

### Example 6

### miRNA Expression Profiling

Samples were obtained from the Banner Sun Health Research Institute and consisted of neuropathologically verified AD, PD, and neurologically normal control subjects. Average expired age was comparable across the three groups: controls (82.1±10 years), AD (81.3±7.7 years) and PD (80.0±5.1 years). Average disease duration was 7.5±4.1 years for AD patients, and 12.6±7.9 years for PD subjects. Mean postmortem interval for all samples was approximately 3.1 hours. In most cases, we were able to analyze one CSF and one SER sample from each subject, hence allowing for direct comparison of miRNA signatures for the two biofluids and reducing sample variability. Supporting the consistency of our results, analysis of variance revealed no significant source of variation in the expression data due to age, gender, or postmortem interval (PMI; FIG. 5).

We conducted miRNA expression profiling of SER and CSF samples using NGS. NGS platforms for miRNA typically require at least 1 μg of total RNA as a starting input. This is problematic for SER and CSF samples which contain low levels of total RNA. We modified a protocol for small RNA deep sequencing for samples with low RNA content and small starting volumes, allowing for miRNA NGS expression profiling from CSF and SER (supra Burgos et al., 2013). We concentrated our down-stream analysis on the 2228 known miRNAs in miRBase (Version 18), out of which 1773 were expressed in at least one CSF sample and 1757 in at least one SER sample. For our analysis, we reduced these numbers to 428 miRNAs in CSF and 414 miRNAs in SER that had a minimum average of >5 read counts. From the 2228 possible mature miRNAs, we removed those that had the same expression patterns across all samples. For example, if has-let-7a-5p_hsa-let-7a-1 and hsa-let-7a-5p_hsa-let-7a-2 were present with the same expression profile, hsa-let-7a-5p_hsa-let-7a-2 was considered redundant and removed from further analysis.

### Example 7

### miRNAs Are Differentially Expressed in CSF and SER of AD Patients

The samples from AD and age-matched non-affected subjects were subsequently analyzed for differential miRNA content. Based on the distribution of total number of mapped reads (sequence reads that align to known mature miRNAs), we set the threshold for removing samples to those with less than 100,000 mapped reads for CSF and less than 60,000 for SER data. Subsequently, we removed m outliers from the following groups: CSF AD (m=5), CSF Control (m=5), SER AD (m=11) and SER Control (m=10). The remaining samples had an average of 2,631,443 reads that mapped to known miRNAs for CSF samples and 1,953,105 mapped read counts for SER samples. To our knowledge these samples represent the largest depth of coverage in any study to date.

A total of 41 miRNAs were determined to have different expression levels between AD CSF (n=62) and Control CSF (n=65), corrected for multiple tests with the Benjamini-Hochberg method and normalized mean>5 mapped reads for each group (FIG. 6).

Sample size for SER consisted of 53 AD, n=50 PD and 62 control subjects. Results were filtered at corrected p-value <0.05 (FIG. 7). We describe only significant differentially expressed miRNAs with an average number of mapped reads greater than 5 and 0.7<FC(log 2) or FC(log 2)<−0.7. Logarithmic base 2 fold change (FC) is relative to the first listed group for each comparison. The overlap of CSF and SER expressed miRNAs for AD compared to neurologically normal control subject analysis consists of two miRNAs, miR-184 and miR-127-3p. The direction of miR-184 and miR-127-3p expression did not correlate between CSF and SER data. It is interesting to note that the miRNAs expressed differently in the CSF were all significantly down-regulated, whereas 85% of the miRNAs identified in SER were up-regulated compared with neurologically normal age-similar controls.

We also examined miRNAs that were different between AD and PD patients (FIGS. 6 and 7). In the CSF, only 1 of the 5 differentially expressed miRNAs between AD and PD subjects was specific to that analysis, and did not overlap with miRNAs that were detectably different in AD compared with control subjects or PD compared with control subjects: 32-5p. In SER, 16 miRNAs had different expression levels when AD and PD subjects were compared, out of which 12 were unique to that analysis and exhibited no overlap with results from CSF with AD or PD compared with control subjects.

### Example 8

### miRNAs are Differentially Expressed in CSF and SER of PD Patients

We surveyed the data sets to detect misregulated miRNAs associated with PD pathology in biofluids. A total of eight PD CSF samples and ten PD SER samples were removed prior to testing for differential expression due to low sample read count.

Seventeen miRNAs were detected as significantly different at corrected p<0.05 between PD CSF (n=57) and Control CSF (n=65) samples (FIG. 6). Interestingly, miR-127-3p, 443, 431-3p, 136-3p and 10a-5p were differentially expressed for both AD compared to Control subjects and PD patients compared with Control subjects, in the CSF.

There were 5 miRNAs differentially expressed in SER samples from PD patients compared to control subjects. The expression levels of miR-338-3p, 30e-3p and 30a-3p were up-regulated in the SER of PD (n=50) subjects, whereas miR-16-2-3p and 1294 were significantly down-regulated (FIG. 7).

These data represent one of the largest data sets to date, examining the miRNAs detectable in cell-free biofluids from patients with neurodegenerative disease, and the first to use NGS to compare the profiles from CSF and SER. We were able to detect differentially expressed miRNAs in CSF and SER. Interestingly, there was minimal overlap in the miRNAs identified in CSF with the miRNAs identified in SER.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method of purifying RNA from a biological sample, the method comprising:
a) mixing the biological sample with a first solution comprising guanidinium isothiocyanate and beta-mercaptoethanol to generate a first mixture;
b) combining a second solution comprising phenol, chloroform, and isoamyl alcohol with the first mixture;
c) centrifuging the first mixture to produce a first aqueous phase, an interface, and an organic phase;
d) removing and saving the first aqueous phase;
e) mixing nuclease-free water with the organic phase and the interface to generate a second mixture;
f) centrifuging the second mixture to produce a second aqueous phase with the interface and the organic phase;
g) removing and saving the second aqueous phase with the first aqueous phase; and
h) concentrating and purifying RNA from the first and second aqueous phases with ethanol-based column chromatography or ethanol precipitation and solubilization of the RNA.

2. The method of claim 1, wherein the acidic pH is between about 4 and about 6.

3. The method of claim 1, further comprising measuring the volume of the first aqueous phase.

4. The method of claim 3, wherein the nuclease-free water is mixed with the organic phase in a volume that is about equal to the volume of the first aqueous phase.

5. The method of claim 1, wherein the second solution consists of 50% phenol, 48% chloroform, and 2% isoamyl alcohol.

6. The method of claim 5, wherein the second solution is mixed with the first mixture in a ratio of 1:1 (v/v).

7. The method of claim 1, wherein the biological sample is selected from the group consisting of cerebrospinal fluid (CSF), whole blood, serum (SER), plasma, urine, saliva, synovial fluid, a bronchioalveolar lavage, a nasal swab, brain tissue, cardiac tissue, bone, skin, a lymph node tissue, and a dental tissue.

8. The method of claim 7, wherein the biological sample is CSF.

9. A method of sequencing RNA in a biological sample, the method comprising:
a) purifying the RNA from the biological sample with the method of claim 1; and
b) sequencing the RNA with next-generation sequencing (NGS).

10. The method of claim 9, wherein the RNA is a small RNA selected from the group consisting of siRNA, miRNA, piRNA, gRNA, snoRNA, and tRNA.

11. The method of claim 10, wherein the small RNA is miRNA.

12. The method of claim 9, wherein the biological sample is selected from the group consisting of CSF, whole blood, SER, plasma, urine, saliva, synovial fluid, a bronchioalveolar lavage, a nasal swab, brain tissue, cardiac tissue, bone, skin, a lymph node tissue, and a dental tissue.

13. The method of claim 12, wherein the biological sample is CSF.

14. The method of claim 9, wherein the NGS comprises ion semiconductor sequencing, cycle sequencing, pyrosequencing, or sequencing using γ-phosphate-labeled nucleotides.

15. A method for diagnosing Alzheimer's disease in a subject, the method comprising:
a) obtaining a biological sample from the subject;
b) determining the expression level of a plurality of miRNAs in the biological sample; and
c) detecting Alzheimer's disease in the subject if there is a significant deregulation of the expression levels of the plurality of miRNAs in the biological sample compared to control values.

16. The method of claim 15, wherein the biological sample is CSF.

17. The method of claim 16, wherein the plurality of miRNAs comprises at least two miRNAs selected from the group consisting of miR-124-3p, miR-138-5p, miR-127-3p, miR-132-3p, miR-127-5p, miR-136-3p, miR-381, miR-101-5p, miR-199b-5p, miR-136-5p, miR-184, miR-181a-5p, miR-598, miR-218-5p, miR-9-3p, miR-769-5p, miR-95, miR-760, miR-181a-3p, miR-181b-5p, miR-488-3p, miR-495, miR-708-3p, miR-874, miR-873-5p, miR-129-5p, miR-181d, miR-139-5p, miR-3200-3p, miR-431-3p, miR-9-5p, miR-326, miR-377-5p, miR-433, miR-323a-3p, miR-134, miR-329, miR-10a-5p, miR-33b-5p, miR-410, and miR-708-5p; and the significant deregulation of the expression levels of the plurality of miRNAs is a decrease in expression compared to control values.

18. The method of claim 15, wherein the biological sample is SER.

19. The method of claim 18, wherein the plurality of miRNAs comprises at least two miRNAs selected from the group consisting of miR-34b-3p, miR-219-2-3p, miR-22-5p, miR-125b-1-3p, miR-1307-5p, miR-34c-5p, miR-34b-5p, miR-887, miR-135a-5p, miR-184, miR-30c-2-3p, miR-873-3p, miR-125a-3p, miR-671-3p, miR-1285-3p, miR-3176, and miR-127-3p; and the significant deregulation of the expression levels of the plurality of miRNAs is a decrease in expression compared to control values.

20. The method of claim 18, wherein the plurality of miRNAs comprises at least two miRNAs selected from the group consisting of miR-182-5p, miR-21-5p, miR-375; and the significant deregulation of the expression levels of the plurality of miRNAs is an increase in expression compared to control values.

21. A method for diagnosing Parkinson's disease in a subject, the method comprising:
a) obtaining a biological sample from the subject;
b) determining the expression level of a plurality of miRNAs in the biological sample; and
c) detecting Parkinson's disease in the subject if there is a significant deregulation of the expression levels of the plurality of miRNAs in the biological sample compared to control values.

22. The method of claim 21, wherein the biological sample is CSF.

23. The method of claim 22, wherein the plurality of miRNAs comprises at least two miRNAs selected from the group consisting of miR-132-5p, miR-485-5p, miR-127-3p, miR-128, miR-409-3p, miR-433, miR-370, miR-431-3p, miR-873-3p, miR-136-3p, miR-212-3p, miR-10a-5p, miR-1224-5p, and miR-4448; and the significant deregulation of the expression levels of the plurality of miRNAs is a decrease in expression compared to control values.

24. The method of claim 22, wherein the plurality of miRNAs comprises at least two miRNAs selected from the group consisting of miR-19a-3p, miR-19b-3p, and let-7g-3p; and the significant deregulation of the expression levels of the plurality of miRNAs is an increase in expression compared to control values.

25. The method of claim 21, wherein the biological sample is SER.

26. The method of claim 25, wherein the plurality of miRNAs comprises miR-16-2-3p and miR-1294; and the significant deregulation of the expression levels of the plurality of miRNAs is a decrease in expression compared to control values.

27. The method of claim 25, wherein the plurality of miRNAs comprises at least two miRNAs selected from the group consisting of miR-338-3p, miR-30e-3p, and miR-30a-3p; and the significant deregulation of the expression levels of the plurality of miRNAs is an increase in expression compared to control values.
